(19) United States
(12) Patent Application Publication
(10) Pub. No.: US 2009/0134056 A1
(43) Pub. Date: May 28, 2009

(54) CHILD IDENTIFICATION KIT FOR HOME USE

(76) Inventor: Kitty Burns, Phoenix, AZ (US)

Correspondence Address: BOYLE FREDRICKSON S.C., 840 North Plankinton Avenue, MILWAUKEE, WI, 53203, US

(21) Appl. No.: 12/324,588

(22) Filed: Nov. 26, 2008

Related U.S. Application Data
(60) Provisional application No. 60/990,102, filed on Nov. 26, 2007.

(51) Int. Cl. B65D 69/00; C12Q 1/68; B05C 11/00; A61B 5/117

(52) U.S. Cl. 206/569; 435/6; 118/264; 427/1

(57) ABSTRACT

A child identification kit suitable for home use includes a chemically treated DNA stabilizing card together with all the materials necessary for taking and storing a reliable DNA together with fingerprints and a photographic of the child in the case of abduction. The materials serve to identify the steps required for complete record creation, supplementing printed instructions and simplifying the task for typical parent.

### CROSS-REFERENCE TO RELATED APPLICATIONS

This application claims the benefit of U.S. provisional application 60/990,102 filed Nov. 26, 2007 and hereby incorporated by reference.

### BACKGROUND OF THE INVENTION

The present invention relates to kits for preserving materials to help in identification of missing children and in particular to a kit suitable for home use.

The U.S. Department of Justice study reported that over 700,000 children (younger than 18) were reported missing in the one-year period of study. In light of these alarming figures, the National Center for Missing and Exploited Children recommends that parents prepare against the possibility of their child becoming missing by taking a color photograph of their child every six months, having their dentists prepare and maintain current dental charts for the child, arranging for the child to have their fingerprints taken by local law enforcement agencies, and keeping a DNA sample of the child, like an old toothbrush, in a paper envelope licked closed by the child. This DNA sample is to be maintained at room temperature in a dry easily accessible place that is far from heat.

It is likely that few if any parents follow this advice. In part this is because the natural tendency of parents to discount the possibility that their child might go missing or to refuse to think about the consequences. Important, too, is the difficulty of coordinating the taking of photographs, discussions with dentists, and contacting law enforcement agencies for fingerprinting. These steps are impractical for most busy parents. Even if these steps were to be completed by the parent, there is substantial question as to whether DNA (for example, on an old toothbrush) stored at room temperature would remain viable (amplifiable) for even a year.

### SUMMARY OF THE INVENTION

The present invention provides a kit collecting in one place all the materials necessary for a parent to prepare a comprehensive child identification record. The kit includes a chemically stabilizing DNA sample card that can safely store DNA for up to 14 years at room temperature. A fingerprinting kit eliminates the need to contact law enforcement agencies for this purpose. A photo module encourages the creation of identification photographs of the child. Finally, a storage container provides a convenient location for keeping these items together and protected.

By greatly simplifying the identification process, the kit significantly increases the possibility that busy parents can practically provide for lasting identification of their children.

### BRIEF DESCRIPTION OF THE DRAWINGS

FIG. 1 is a perspective view of the kit of the present invention showing its various elements as may be contained in a compact storage container.

### DETAILED DESCRIPTION OF THE INVENTION

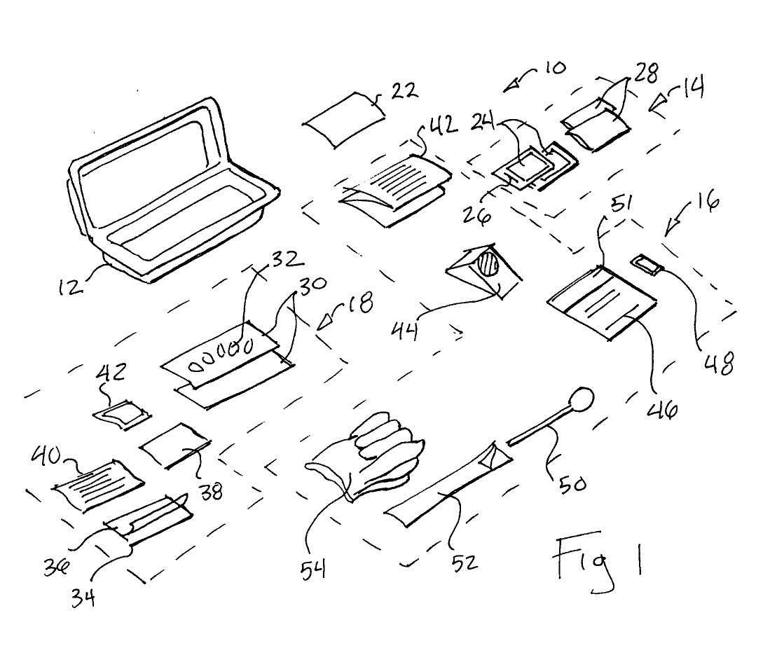

Referring now to FIG. 1, a home child identification kit of the present invention includes a compact storage container 12 such as a plastic clam shell that may serve to package and ship the kit as well as to hold the elements of the kit in a collected form after use in the home. The container 12 preferably is resealable, for example, by a compression fitting of the halves of the clamshell together, or other means, to provide protection of its contents away from environmental contamination both before and after use. A paperboard insert, not shown, may lie in the container and serve to provide identifying information about the kit for retail sales and to protect the contents of the kit before and after use against environmental contamination and light.

The contents of the kit include three principal parts: a photographic module 14, a DNA module 16, and a fingerprint module 18 together with a mission statement card 22, the latter intended to remind users of the importance of this activity and promote the user's encouragement of other parents to take these important steps.

The photographic module 14 may include two photo-mounting cards 24, one being a sample and the other intended for use by the parent. The photo-mounting cards 24 provide a place for color photographs of the children and blanks 26 in order to record the children's age. The obverse side of each photo-mounting card 24 provides a place for the thumbprint of the child to provide redundancy with the fingerprint module 18. The photo-mounting card 24 both reinforces and protects a color photograph (not shown) mounted to the photo-mounting card 24 and encourages the use of a photograph sized amply to provide suitable resolution and detail.

Plastic sleeves 28 are provided into which the photo-mounting cards 24 may be slid to further protect the attached photographs. The sample photo-mounting card 24 is preprinted with a thumbprint and a sample picture to provide guidance to the parent in completing this step.

The fingerprint module 18 provides two fingerprint-recording cards 30. Again, one fingerprint-recording card 30 is a sample pre-printed with fingerprints 32 to show the parent what the final fingerprint should look like. One side of each fingerprint-recording card 30 provides blanks to record the child's name and age for the child's right-hand and the obverse side of each fingerprint-recording card 30 provides a similar space for fingerprints of the left hand.

The fingerprint module 18 includes all the elements necessary for an inexperienced user to create good-quality fingerprints when following included instructions 40. These elements include a pre-inked fingerprint pad 34 having a peel-away plastic covering 36 avoiding a need for liquid inks. Also included is a practice paper 38 for practicing obtaining good fingerprints and premoistened wipe 42 in a foil package for cleaning the child's fingers after completion of this process. Again, the intent is to provide all materials necessary for the parent to create fingerprints and thus to make practical this important identification process which otherwise, simply for its inconvenience, might not be completed.

The DNA module 16 includes separate instructions providing simple cartoons and text to guide the parent through this process. Significantly the DNA module 16 provides a chemically treated DNA stabilizing card 44 containing chemicals that lyse cells, denature proteins, and protect nucleic acids from nucleases, oxidation, and UV damage. A suitable stabilizing card 44 is commercially available from Whatman of London, UK under the trade name “Indicating FTA® Micro Card” and as described in U.S. Pat. No. 5,985,327 hereby incorporated by reference.

The stabilizing card 44 provides a color indicator that changes from pink to white when a sample is applied to the stabilizing card 44, simplifying use by a layperson. DNA may be stored on this stabilizing card 44 at room temperature for over 14 years.

The stabilizing card 44 may be sealed with a clear sticker (not shown) and placed inside a multi-barrier pouch 46 after DNA is sampled and stored on the stabilizing card 44, the multi-barrier pouch 46 providing multiple plastic film and foil layers to protect the stabilizing card 44 from gas or liquid contamination. The outer surface of the multi-barrier pouch 46 provides a label having blanks for the entry of the child's name, age, and special information (for example, scars, hair, and eye color) to be recorded by the parent. A desiccant pack 48 is also provided that can be inserted into the multi-barrier pouch 46 before the multi-barrier pouch 46 is sealed by a self-contained adhesive strip 51.

In order to prevent contamination of the DNA sample placed on the stabilizing card 44, the DNA module 16 also includes a sterile swab 50 having a foam tip and stored in a sterile package 52. The DNA module 16 also provides sterile gloves 54 for use by the parent in this process. Following the instructions 40, DNA is typically collected from the cheeks, teeth and under the tongue of the child with the sterile swab 50 and then applied to the stabilizing card 44.

By collecting these materials and providing clear instructions, convenient home child identification can be performed making such identification far more likely than if the parent is required to go to multiple professionals to obtain the necessary components of a complete identification.

Once the identification process is completed by the parent, the fingerprint-recording card 30, photo-mounting cards 24 in sleeve 28, and multi-barrier pouch 46 containing the stabilizing card 44 and desiccant pack 48 are placed back in the container 12 and the container 12 is closed and stored in a safe place in the home or where the parent can obtain ready access to these materials. By collecting these materials in one location, it is likely that a parent can quickly obtain access to these materials in the event their child should become missing.

It should be understood that the invention is not limited in its application to the details of construction and arrangements of the components set forth herein. The invention is capable of other embodiments and of being practiced or carried out in various ways. Variations and modifications of the foregoing are within the scope of the present invention. It also being understood that the invention disclosed and defined herein extends to all alternative combinations of two or more of the individual features mentioned or evident from the text and/or drawings. All of these different combinations constitute various alternative aspects of the present invention. The embodiments described herein explain the best modes known for practicing the invention and will enable others skilled in the art to utilize the invention.

I claim:

1. A kit for home child identification comprising:
a resealable storage container holding:
a DNA module having:
(1) a card chemically treated for the preservation of the DNA at room temperature for greater than five years;
(2) a pouch for storing this card and sealable against light and contamination;
(3) a desiccant for insertion into the pouch prior to its ceiling;
(4) a sterile packed swab for sampling DNA from a child's mouth;
(5) sterile gloves;
a fingerprint module having:
(1) a fingerprint ink pad;
(2) cards for receiving fingerprints taken using the fingerprint inkpad.

2. The kit for home child identification of claim 1 further including a photograph card and a protective plastic sleeve for holding the photograph card and wherein the card providing printed indicia showing a location for placement of a photograph of a child and a location for entry of the child's age marked by a caption denoting age.

3. The kit for home child identification of claim 2 wherein the photograph card further includes a location for a thumbprint and a caption for the location denoting a thumbprint on an opposite side from the printed indicia showing a location for placement of a photograph.

4. The kit for home child identification of claim 3 further including a first sample card matching the card for receiving fingerprints showing sample fingerprints and a second sample card matching the photograph card showing a sample child's picture and age entered into the location for entry of the child's age.

5. The kit for home child identification of claim 4 further including a label on the outside of the pouch providing captioned locations for a child's name and age.
